(19) United States
(12) Patent Application Publication
(10) Pub. No.: US 2009/0216291 A1
(43) Pub. Date: Aug. 27, 2009

(54) SYSTEM AND METHOD FOR DETECTING ELECTRIC EVENTS IN CHAMBERS OF A HEART

(76) Inventors: Nils Holmström, Järfälla (SE); Anders Björling, Solna (SE)

Correspondence Address: SCHIFF HARDIN, LLP;PATENT DEPARTMENT, 6600 SEARS TOWER, CHICAGO, IL, 60606-6473, US

(21) Appl. No.: 12/160,908

(22) PCT Filed: Jan. 31, 2006
(86) PCT No.: PCT/SE2006/000143

§ 371 Date: Nov. 5, 2008

(51) Int. Cl. A61N 1/368; A61B 5/0402

(52) U.S. Cl. 607/17; 600/509

(57) ABSTRACT

In a system and method for detecting electrical cardiac events, and a heart stimulator embodying such a system, cardiac events are detected in respective chambers of a heart by sensing electrical signals in at least two different chambers of the heart and forming a difference signal from the sensed signals, and using the difference signal to automatically distinguish between events originating from one of the chambers and events originating in another of the chambers. At least one of the sensed signals is sensed in a coronary vein on the left atrium or the left ventricle.

### BACKGROUND OF THE INVENTION

1. Field of the Invention

The present invention relates to a system for detecting electric cardiac events in chambers of a heart of the type having leads with electrodes adapted for implantation in at least two different chambers of the heart for sensing electric signals therein, and a difference former provided to supply a difference signal, formed of the signals sensed by the electrodes, to an event detector, arranged to distinguish, from the difference signal, between events originating from one of the chambers from events originating from the other one of the chambers. The invention also relates to a corresponding method and a heart stimulator embodying such a system.

2. Description of the Prior Art

Multi-chamber sensing system for use in e.g. multi-chamber pacing systems, need to sense electrical cardiac events, like P and R-waves, at several positions inside or outside of the heart muscle. Leads provided with one or more electrodes are used for this purpose.

Bipolar sensing of both atrial and ventricular events is thus described in U.S. Pat. No. 5,800,468.

Differential sensing between unipolar electrode leads located in the right atrium and the right ventricle is disclosed in U.S. Pat. No. 5,871,507.

In United States Patent Application Publication No. 2001/0049543 biventricular stimulation and capture monitoring are described. For capture detection cross-chamber sensing electrode configurations are used. Multipolar leads are used and different electrodes are used for stimulation and sensing. Sensing between a right ventricular ring electrode and a left atrial ring electrode is described as well as between right and left ventricular ring electrodes.

To be able to position electrode leads in the coronary veins for stimulation and sensing on the left ventricle and atrium of as many patients as possible with varying anatomy, it is important that the leads are as thin and flexible as possible. Leads with at least an unipolar distal end portion are easier to make thin and flexible and are therefore normally easier to implant in the coronary veins. However, when using an unipolar electrode for sensing with the stimulator or sensing apparatus case as indifferent electrode, the signal-to-noise ratio often becomes unacceptable low.

### SUMMARY OF THE INVENTION

An object of the present invention is to solve this problem and make detection of capture and loss of capture possible in all the chambers of the heart, also of the left atrium and left ventricle without using the above-mentioned conventional unipolar sensing which often results in a too low signal-to-noise ratio as mentioned.

The above object is achieved in accordance with the present invention by a system for detecting electrical cardiac events in chambers of a heart, having multiple leads each provided with at least one electrode, the lead being respectively configured for implementation in at least two different chambers of the heart for sensing electrical signals in those chambers, and a difference former connected to the leads that emits a difference signal formed by the respective signals sensed by the electrodes of the respective leads, and an event detector, supplied with the different signals, that distinguishes between electrical events originating in one of the chambers and electrical events originating in the other of the chambers, wherein at least one of the leads is adapted for implantation in a coronary vein of the art for sensing a left atrial signal or a left ventricular signal.

The above object also is achieved by a cardiac stimulator embodying such a detection system, as well as by a method that includes sensing a left atrial signal or a left ventricular signal using a lead configured for implantation in a coronary vein of a heart.

Thus, according to the present invention detection of electric cardiac events is possible in any one of the four chambers of the heart while avoiding the disadvantage of the above discussed, previously used technique for unipolar sensing by performing the sensing between electrodes located within or on the heart.

According to an advantageous embodiment of the system according to the invention the leads adapted for implantation in a coronary vein on the left side of the heart are unipolar. The use of unipolar left side leads involves several important advantages. Unipolar leads are thinner than bipolar leads, they are softer and more flexible at the distal end portion, have a simpler mechanical construction and a better longevity. An unipolar lead can be more easily bent distally using a pre-bent or steerable stylet which is important for making passage of sharp bends in coronary vessels possible. It is also easier to slide a unipolar lead over a sharply bent guidewire inside a coronary vein and to retract the guidewire after positioning the lead tip in a proper position. Bipolar leads, on the other hand, are often too thick for implantation in all the coronary veins which can be candidates for e.g. left ventricular stimulation and sensing. It can for instance be impossible to pass a sharp bend because the distal end of the bipolar lead is too stiff. The advantages of bipolar leads are that they normally give a higher signal-to-noise ratio, and bipolar stimulation reduces the risk of muscular stimulation at the stimulator case. Bipolar sensing can, however, sometimes be difficult when an action potential is passing both electrodes simultaneously and the signals are similar. In such a case unipolar sensing is preferred. Phrenicus stimulation also often requires attention at left ventricular pacing, since bipolar pacing on the left ventricle increases the risk of phrenicus stimulation because both the tip and the ring are involved. With this embodiment of the invention the risk of phrenicus stimulation is reduced compared to normal bipolar or unipolar stimulation which also can result in pectoral muscle stimulation when higher stimulation amplitudes are needed. In summary, with the present embodiment the physician will be more free to find an optimal location of the implanted lead on the left side of the heart, which can result in a higher phrenicus stimulation threshold, better hemodynamics and low stimulation threshold, while not losing the good properties of using bipolar leads.

When stimulating between tip electrodes located in the left and right ventricles it is difficult to detect evoked response between the tips, because the difference signal can be small or totally disappear in case of capture on both the ventricles. In this case a detection can be erroneously interpreted as loss of capture in both ventricles instead of capture in both ventricles. According to other advantageous embodiments of the system according to the invention, the event detector is therefore arranged to distinguish, after bi-ventricular stimulation, between capture and non-capture from the difference signal, formed of signals sensed by an electrode positioned in the left ventricle and by a differently positioned electrode on the right side of the heart, the differently positioned electrode on the right side of the heart preferably being a ring or coil electrode. In this way there will be a time lag between evoked response signals sensed in the right and left side of the heart depending on the distance between the ventricular tip electrode and the differently positioned electrode on the right side of the heart and on the propagation velocity of the action potential in the myocardium, when pacing takes place simultaneously on both sides of the heart and capture of both ventricles is obtained.

According to still another advantageous embodiment of the system according to the invention the electrodes are adapted for implantation in a coronary vein on the left atrium, and in a coronary vein on the left ventricle or in the right ventricle respectively. Sensing between a tip electrode in the left atrium and a tip electrode in the left ventricle can be an attractive alternative. It is possible to distinguish between a P-wave and a R-wave from the direction of the rapid voltage change when the action potential passes the electrodes. By using an electrode in one of the ventricles as indifferent electrode when sensing P-waves in the left atrium interference of P-waves in the right atrium is avoided.

### BRIEF DESCRIPTION OF THE DRAWINGS

FIG. 1 schematically illustrates a bi-ventricular pacemaker system with bi-ventricular sensing.

FIG. 2 shows intracardiac electrograms (IEGMs) between right and left ventricular electrode tips recorded in heart failure patients.

FIG. 3 shows an IEGM measure between right and left ventricular electrode tips for a heart failure patient having Left Bundle Branch Block (LBBB).

FIG. 4 shows the IEGM signal in FIG. 3 differentiated.

FIG. 5 shows two examples of the signal sensed between the right and left ventricular electrode tips.

FIG. 6 shows the derivative of the signals in the examples of FIG. 5.

FIG. 7 illustrates simplified curves representing expected responses after pacing.

FIG. 8 schematically illustrates an embodiment of the invention using a bipolar electrode in the right ventricle and a unipolar electrode in the left ventricle.

FIG. 9 schematically illustrates an associated detector, and evoked response signals that occur upon capture of both ventricles.

FIG. 10 schematically illustrates an embodiment of the invention including an additional unipolar electrode lead implanted in a coronary vein on the left atrium.

FIG. 11 shows the difference signal sensed between the electrodes on the left side of the heart.

FIG. 12 schematically illustrates a four-chamber pacemaker system having unipolar leads on the left side of the heart and bi-polar leads on the right side of the heart.

### DESCRIPTION OF THE PREFERRED EMBODIMENTS

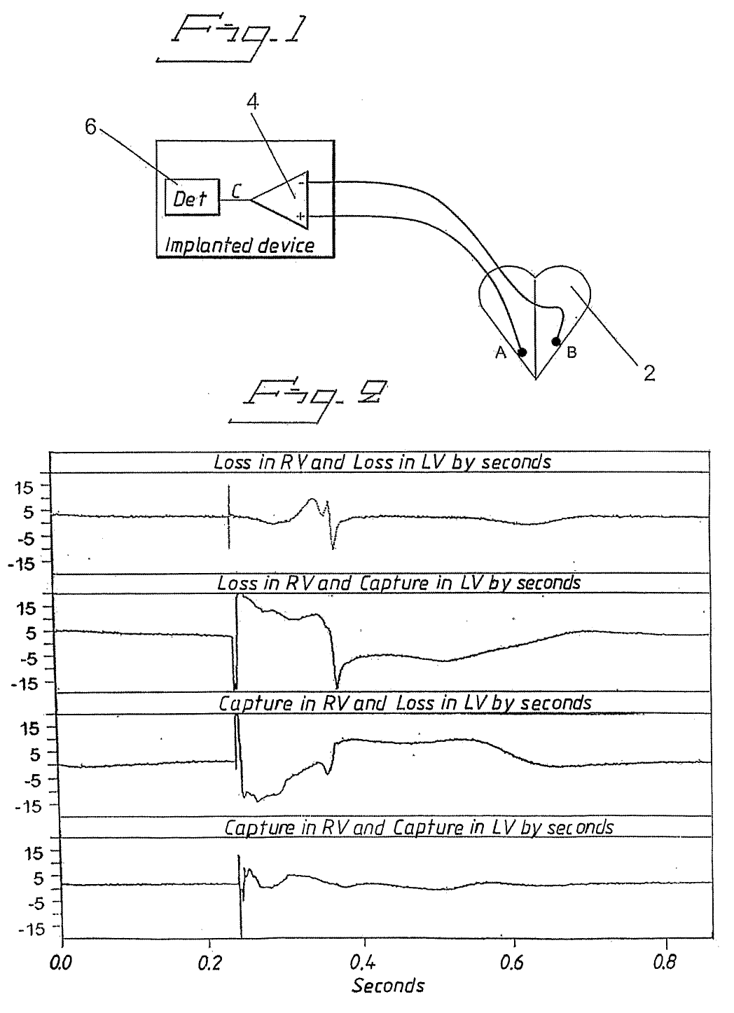

FIG. 1 shows schematically an implanted device comprising a bi-ventricular pacemaker system having bi-ventricular sensing. One electrode A is implanted in the RV of a heart 2 and another electrode B is implanted in a coronary vein on the LV. The sensed difference signal C obtained as output signal from the difference forming means 4 is supplied to an event detector 6 for determining both origin and time of electric cardiac events.

FIG. 2 shows IEGMs recorded between tip electrodes in RV and LV, cf. FIG. 1, in heart failure patients having LBBB after bi-ventricular pacing between the tip electrodes.

The curve at the bottom of FIG. 2 shows that capture in both ventricles results in a very small IEGM signal because the evoked responses practically eliminate each other since the signal amplitudes are about the same. The measured RV evoked response signal amplitude amounted to 13.1 mV and the LV evoked response signal amplitude to 14.2 mV.

The second curve from below in FIG. 2 illustrates a situation with capture in RV and loss of capture in LV. Due to the loss of capture in LV an intrinsic R-wave is generated after about 120 msec.

The second curve from the top of FIG. 2 illustrates a situation with capture in LV and loss of capture in RV. An intrinsic right ventricular R-wave appears after about 120 msec.

The curve at the top of FIG. 2 illustrates a situation with loss of capture in both RV and LV. The two intrinsic R-waves appear almost simultaneously.

From FIG. 2 it can be seen that with these measurements it is possible to distinguish the four situations illustrated from each other. A predetermined negative threshold value is applied in a time window after the pacing pulse for comparison with the sensed IEGM signal for indicating capture in RV and loss of capture in LV. Similarly, a predetermined positive threshold value is applied in a time window after the pacing pulse for comparison with the sensed IEGM signal for indicating capture in LV and loss of capture in RV. Detection of no signal passage of neither the positive nor the negative threshold value means capture of both ventricles or loss of capture for both ventricles.

One way of distinguishing between capture of both ventricles and loss of capture for both ventricles is to perform a separate R-wave detection. In U.S. Pat. No. 6,148,234 an R-wave detector is described which is enabled from the delivery of a stimulation pulse to the end of the heart refractory period, a period of normally about 300 msec. If R-waves are detected in this period there has been loss of capture in one of the ventricles.

Another way of distinguishing capture of both ventricles from loss of capture for both ventricles is to study separately the signals from RV and LV respectively in case of detection of no difference signal.

Still other possibilities of distinguishing capture of both ventricles from loss of capture for both ventricles will be described below.

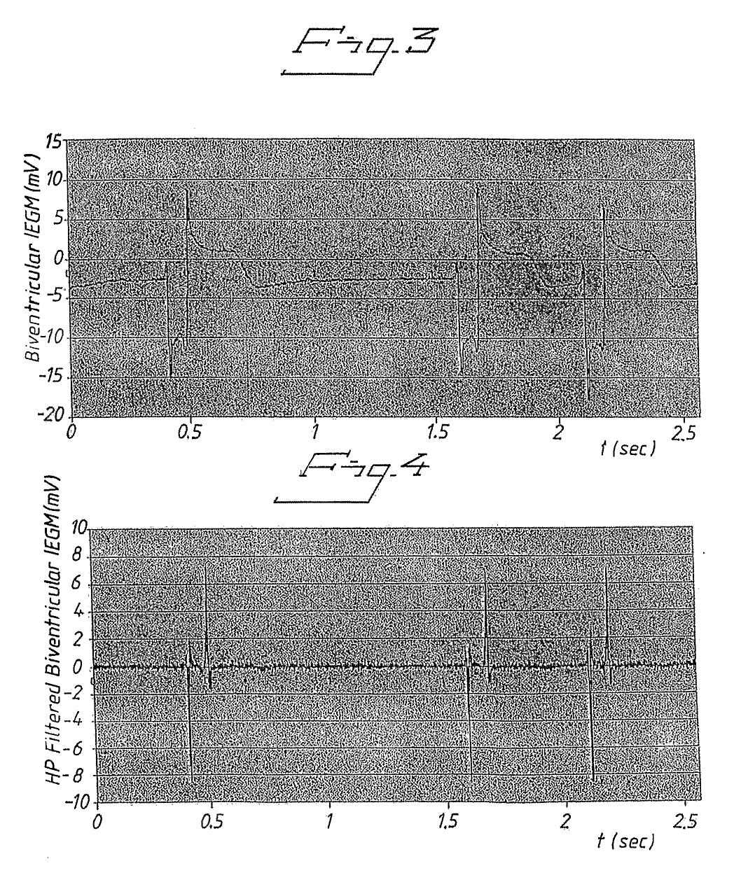

Sensing between tip electrodes in RV and LV gives almost a bipolar suppression of noise signals. In FIG. 3 the intrinsic tip to tip IEGM signal from a heart failure patient having LBBB is shown. The QRS complex is a fast negative deflection signal relating to RV. After about 80 msec a fast positive signal deflection appears relating to LV.

By high pass filtering or differentiating the signal in FIG. 3 a curve as shown in FIG. 4 is obtained representing the derivative of the signal in FIG. 3. Negative peaks in the derivated signal corresponds to R-waves in RV and positive peaks to R-waves in LV. A threshold detector can then be easily used for detecting the R-waves.

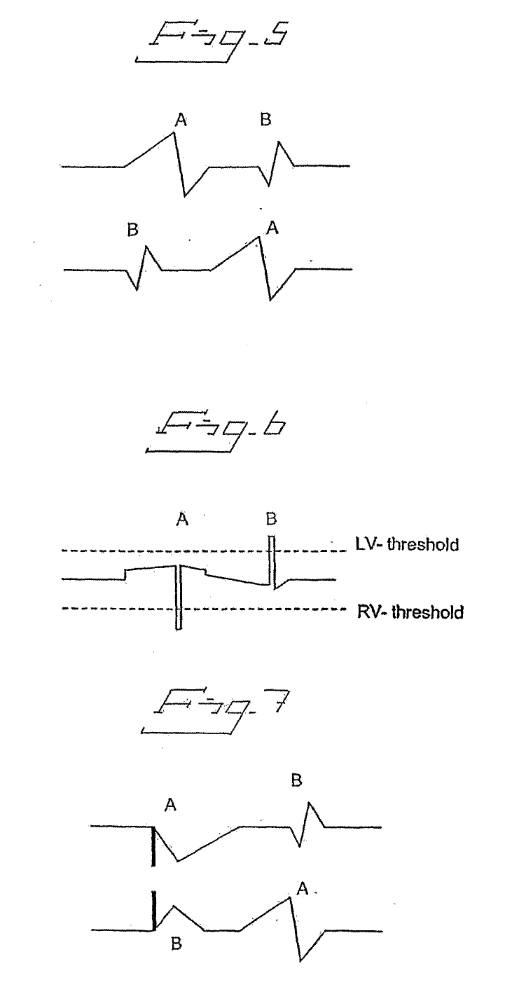

FIG. 5 shows a simplified representation of difference signals sensed between the ventricular tip electrodes A and B in FIG. 1. The upper curve in FIG. 5 shows that the R-wave in RV appears earlier than the R-wave in LV. The lower curve illustrates a situation in which the R-wave in LV comes first.

As described above a suitable high pass filter can be used for making it easier to distinguish between different cardiac events. High pass filtering will enhance the fast derivatives of the IEGM, cf. the discussion above of FIGS. 3 and 4. This is illustrated in FIG. 6 which shows in a simplified way the derivative dC/dt of the difference signal C. This curve clearly reveals the origins of the cardiac events and detection of them is easily carried out by comparisons with LV- and RV-thresholds.

Another technique for distinguishing between cardiac events originating from one of said chambers from events originating from the other one comprises morphology analysis of the difference signal. When the action potential passes the electrode the potential is first raised, thereafter a fast negative slope occurs followed by a return to the baseline. Depending on which (positive or negative) inputs of the difference forming means 4 the electrodes A and B are connected to, see FIG. 1, the difference signal C is inverted or not, cf. FIG. 5.

FIG. 7 shows simplified graphs of evoked response signals after capture in case of bi-ventricular stimulation. The upper graph illustrates capture in RV and an (inverted) R-wave in LV, and the bottom graph (inverted) capture in LV and an R-wave in RV. Thus, cardiac events originating from one of the heart chambers can also be distinguished from events originating from the other chamber by analysis of the polarity of the difference signal. Alternatively the cardiac event detector can comprise integrating means for time integrating the difference signal in a predetermined time window to distinguish between events originating from one of the chambers from events originating from the other one of the chambers by analysis of the integrated difference signal value.

In this connection it could be noted that bi-ventricular pacing between the tip electrodes in RV and LV can create capture in both ventricles simultaneously by one single pacing pulse. Normally the anodic stimulation threshold is higher than the cathodic one. Therefore it is possible to change pacing polarity so that the electrode with the higher stimulation threshold is stimulated cathodically (negatively) and the electrode with the lower stimulation threshold—normally the tip electrode in RV—is stimulated anodically (positively). The transventricular (right ventricular tip electrode to left ventricular tip electrode) stimulation threshold will then be lower. Transventricular pacing will also be associated with a low risk of creating phrenicus nerve stimulation, because the pacing current flows through the heart and not to the pacemaker case or along the lead.

As explained above it is preferred to use unipolar electrodes on the left side of the heart. It is well known to use unipolar as well as bipolar electrodes in the right side of the heart. In this connection it could be an advantage to use bipolar electrodes in right atrium and right ventricle.

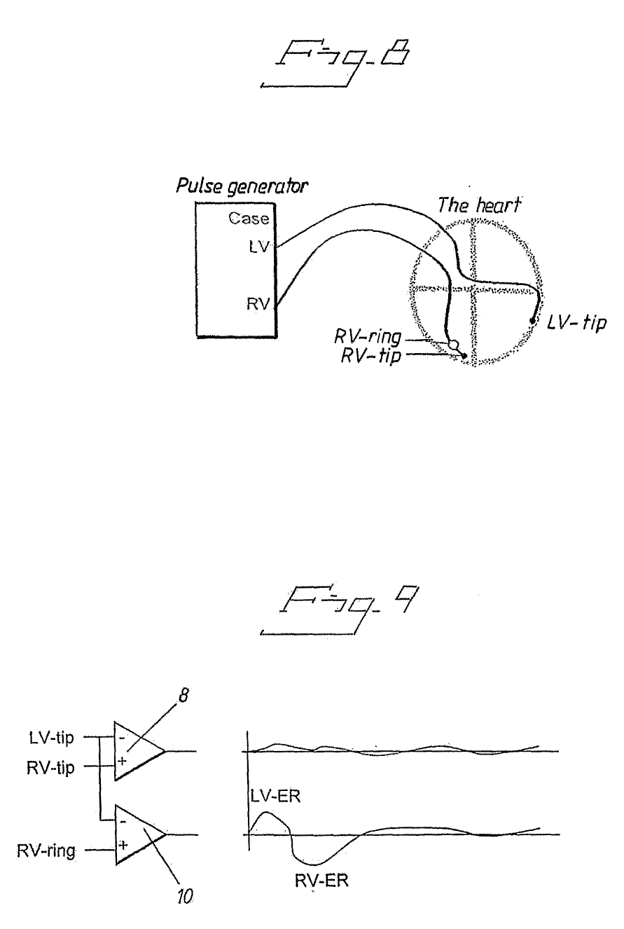

Above sensing of evoked response between tip electrodes in RV and LV respectively when pacing between the two tip electrodes has been described. As explained above, when detecting evoked response, ER, between ventricular electrode tips in this way the difference signal can be very small or totally disappear when having capture on both ventricles or loss of capture on both ventricles, see FIG. 9 upper curve, which shows the difference signal in case of capture on both ventricles. To the left in FIG. 9 difference former in the form of differential detectors 8, 10 are shown. In this case the detection can erroneously be misinterpreted as having loss on both tip electrodes. A way of eliminating this drawback consists in sensing ER between the electrode tip on LV and a differently positioned electrode on the right side of the heart. FIG. 8 thus shows a stimulation pulse generator connected to a patient's heart by a bi-ventricular electrode having a ring electrode, RV-ring, and a tip electrode, RV-tip, in RV and an unipolar electrode, LV-tip, in a coronary vein on the LV. In this embodiment ER is sensed between RV-ring and LV-tip. There will then be a time lag between the occurrence of the ER signal on the electrodes of about 10-30 msec, depending on the distance between tip and ring and the propagation velocity of the action potential in the myocardium, when a single differential pacing pulse supplied by the tip electrodes is used for capturing both ventricles. This is illustrated by the lower graph in FIG. 9. FIG. 9 also shows how the electrode leads of FIG. 8 are connected to the differential detectors 8 and 10.

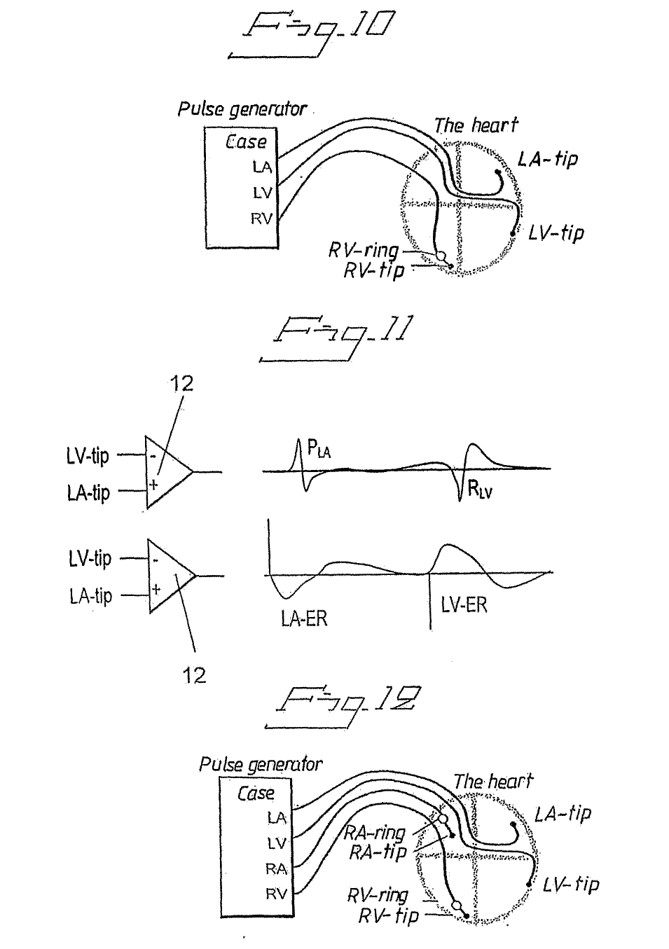

FIG. 10 shows schematically a left atrial lead implanted in a coronary vein on the LA of the heart. The left atrial unipolar lead is suitably implanted in a coronary vein with an entrance from the coronary sinus called “oblique vein of the left atrium”. In this example a stimulator pulse generator is connected to a heart by a bipolar lead in RV and unipolar leads implanted in coronary veins on the left atrium, LA-tip, and ventricle, LV-tip. In this setup sensing the difference signal between LA-tip and LV-tip is suitable.

In the left part of FIG. 11 it is shown how the electrodes LV-tip and LA-tip are connected to a differential detector 12. A P-wave P_{LA} can be distinguished from an R-wave R_{LV} by the direction of the rapid voltage change, which occurs when the action potential passes the electrodes, see the upper graph in FIG. 11. The lower graph in FIG. 11 illustrates sensed left atrial ER signal and left ventricular ER signal following stimulation. Left atrial pacing should be made between the LA-tip electrode and a ring or coil electrode in the heart to reduce the risk for phrenicus stimulation, which can occur in case of unipolar stimulation with the pulse generator case used as indifferent electrode. Differential stimulation between the electrodes LA-tip and LV-tip on the left side of the heart should be avoided.

FIG. 12 shows an example of four chamber pacing. A pacemaker pulse generator is connected to a heart by thin unipolar leads with an LA-tip electrode and an LV-tip electrode implanted in coronary veins on the left side of the heart, and by bipolar leads with RA-tip electrode, RA-ring electrode, RV-tip electrode and RV-ring electrode in the right side of the heart. Left atrial pacing is preferably applied between the electrodes LA-tip and RA-tip or RV-ring, and left ventricular pacing between the electrodes LV-tip and RV-ring or RA-ring. There are several possibilities for sensing P-waves in the left atrium. In the preferred alternative an electrode in one of the ventricles is used as indifferent electrode in order to avoid interference of P-waves from the right atrium. Similarly for sensing R-waves in LV an atrial electrode is preferably used as indifferent electrode.

Although modifications and changes may be suggested by those skilled in the art, it is the intention of the inventors to embody within the patent warranted heron all changes and modifications as reasonably and properly come within the scope of their contribution to the art.

We claim as our invention:

1-22. (canceled)

23. A system for detecting electrical cardiac events in chambers of a heart, comprising:
a plurality of leads, each carrying at least one electrode, configured for implantation respectively in at least two different chambers of a heart for sensing electrical signals in said at least two different chambers;
a difference former electrically connected to said plurality of leads and configured to form a difference signal from respective signals sensed by the electrodes carried by the respective leads;
an event detector supplied with said difference signal and configured to distinguish, from said difference signal, between events originating in one of said at least two different chambers and events originating in another of said at least two different chambers; and
at least one of said leads being configured for implantation in a coronary vein for sensing a left atrial signal or a left ventricular signal.

24. A system as claimed in claim 23 wherein said at least one of said leads that is configured for implantation in a coronary vein is a unipolar lead.

25. A system as claimed in claim 23 wherein said lead configured for implantation in a coronary vein is configured for implantation in a coronary vein on the left side of the heart, and wherein another of said leads is configured for implantation in one chamber on the right side of the heart.

26. A system as claimed in claim 23 wherein said leads consist of a lead configured for implantation in a coronary vein on the left atrium of the heart, and a lead configured for implantation in a coronary vein on a left ventricle of the heart or in the right ventricle of the heart.

27. A system as claimed in claim 23 wherein said event detector is configured to distinguish between said events by making a morphology analysis of said difference signal.

28. A system as claimed in claim 23 wherein said event detector is configured to distinguish between said events by analyzing a polarity of said difference signal.

29. A system as claimed in claim 23 wherein said event detector comprises an integrator that integrates said difference signal over time within a predetermined time window, to obtain an integrated difference signal value, and is configured to distinguish between said events by analysis of said integrated difference signal value.

30. A system as claimed in claim 23 wherein said event detector comprises a differentiator that produces a derivative of said difference signal, and a comparator that compares said derivative with a predetermined threshold to obtain a comparison result, and wherein said event detector is configured to distinguish between said events dependent on said comparison result.

31. A system as claimed in claim 23 wherein said event detector is configured to identify an origin and a time of occurrence of said events.

32. An implantable cardiac stimulator comprising:
a stimulation pulse delivery system configured to interact with a heart to deliver stimulation pulses to cardiac tissue of the heart;
a plurality of leads, each carrying at least one electrode, configured for implantation respectively in at least two different chambers of a heart for sensing electrical signals in said at least two different chambers; a difference former electrically connected to said plurality of leads and configured to form a difference signal from respective signals sensed by the electrodes carried by the respective leads, an event detector supplied with said difference signal and configured to distinguish, from said difference signal, between events originating in one of said at least two different chambers and events originating in another of said at least two different chambers, and at least one of said leads being configured for implantation in a coronary vein for sensing a left atrial signal or a left ventricular signal; and
a control unit configured to operate said stimulation pulse delivery system dependent on said events detected by said system for detecting electrical cardiac events.

33. A cardiac stimulator as claimed in claim 32 comprising an R-wave detector connected to said event detector, said R-wave detector being enable by delivery of a stimulation pulse at an end of a refractory period of the heart, and wherein said event detector is configured to distinguish, from an output signal from said R-wave detector, loss in both ventricles from capture in both ventricles.

34. A cardiac stimulator as claimed in claim 32 wherein said at least one lead configured for implantation in a coronary vein is configured for implantation in a coronary vein on the left side of the heart, and wherein another of said plurality of leads is configured for implantation in one chamber on the right side of the heart, and wherein said event detector is configured to distinguish, from said difference signal after biventricular stimulation by said stimulation pulse delivery system, between capture and non-capture.

35. A cardiac stimulator as claimed in claim 34 wherein said lead configured for implantation in one chamber on the right side of the heart carries an electrode selected from the group consisting of a ring electrode and a coil electrode.

36. A method for detecting electrical cardiac events in chambers of a heart, comprising the steps of:
sensing electrical signals respectively in at least two different chambers of the heart, including sensing with an electrode located in a coronary vein on the left atrium or on the left ventricle, to obtain respective sensed signals;
automatically electronically forming a difference signal from said sensed signals; and
using said difference signal, distinguishing electrical events originating from one of said chambers from electrical events originating in another of said chambers.

37. A method as claimed in claim 36 comprising sensing said electrical signals in one chamber on the right side of the heart and in a coronary vein on the left side of the heart.

38. A method as claimed in claim 37 comprising bi-ventricularly stimulating the heart, sensing electrical signals from a left ventricular sensing point and from a sensing point on the right side of the heart, respectively, and, from said difference signal, automatically electronically distinguishing between capture in both ventricles and non-capture in both ventricles.

39. A method as claimed in claim 36 comprising sensing electrical signals on the left atrium and in one of the ventricles.

40. A method as claimed in claim 36 comprising distinguishing said events by making a morphology analysis of said difference signal.

41. A method as claimed in claim 36 comprising distinguishing said events by analyzing a polarity of said difference signal.

42. A method as claimed in claim 36 comprising integrating said difference signal over time to obtain a time integrated difference signal value, and distinguishing said events by analysis of said time integrated difference signal value.

43. A method as claimed in claim 36 comprising differentiating said difference signal to obtain a derivative of said difference signal, comparing said derivative to a predetermined threshold value to obtain a comparison result, and distinguishing said events dependent on said comparison result.

44. A method as claimed in claim 36 comprising identifying an origin and a time of occurrence of said events from the sensed electrical signals.
